(19) United States
(12) Patent Application Publication
(10) Pub. No.: US 2006/0045897 A1
(43) Pub. Date: Mar. 2, 2006

(54) Occlusive articles for wart treatment

(75) Inventor: John P. Kennedy, Sarasota, FL (US)

Correspondence Address: Law Offices of John D. Gugliotta, PE, Esq.;202 Delaware Building, 137 South Main Street, Akron, OH, 44308, US

(73) Assignee: Aso Corporation

(21) Appl. No.: 10/926,692

(22) Filed: Aug. 27, 2004

(51) Int. Cl. A61K 9/70

(52) U.S. Cl. 424/402; 424/443

(57) ABSTRACT

An occlusive article is disclosed which functions to provide an occlusive or partially occlusive environment for the effective treatment of any wart where occlusive therapy may be practically administered.

### RELATED APPLICATIONS

There are no previously filed, nor currently any co-pending applications, anywhere in the world.

### BACKGROUND OF THE INVENTION

1. Field of the Invention

The present invention relates generally to wart treatment, and more particularly, to occlusive articles for wart therapy.

2. Description of the Related Art

A wide variety of warts are found on human skin and are caused by the human papilloma virus (HPV). The following types of warts are caused by HPV: Verruca vulgaris (the common wart); plantar warts, mosaic warts, planar warts, plantar warts, palmar warts, and condyloma accuminatum (venereal warts).

As obdurate and unsightly as its name, Verruca vulgaris inconveniently finds temporary niches on thousands of people, many of whom avoid revealing its unwelcome presence. It wreaks havoc in the cracks and crevasses of skin, most often causing cosmetic embarrassment and pain.

Throughout the years a number of therapies have been developed for treating warts such as surgical excision, laser vaporization, topical chemical treatment, and cryotherapy (blister-producing method applying liquid nitrogen). Cryotherapy, independent or in conjunction with a secondary methodology, has evolved as the professional treatment of choice for most practicing clinicians. However, freezing warts with liquid nitrogen results in severe pain and discomfort and is frightening to children and young teens.

Adhesive tape has been a non-FDA approved, home remedy for warts for years. This methodology was historical spurned by the professional clinical community. However, recent studies have been published that objectively investigated the efficacy of occlusive therapy for warts using common industrial duct tape. The study produced dramatic results. Resolution of the wart occurred in 22/26 (85%) of patients in the duct tape arm vs. 15/25 (60%) in the cryotherapy arm (p=0.5) within the follow up period of 2 months. Accordingly many dermatologist and family physicians have now added occlusive therapy to their therapeutic regimens.

While the efficacy of occlusive therapy has received strong acceptance in the professional and patient communities, there remain many negative attributes from the perspective of the end user. First, the cosmetic appeal of many tapes is socially distracting. Especially for peer conscious teens, occlusive articles in the form of common duct, or otherwise industrial, tape tends to draw the scrutiny and often harassment of classmates. Aside from cosmetics, the industrial tape is cut to size and shape by the user. This is hardly a reproducible process and often attenuates the adhesion of the industrial tape by inadvertent contact with skin oils and particulate debris. Strong adhesion is critical for efficacy as the tape must stay on for approximately 5-7 days. Another disadvantage is the lack of commercially available pre-cut shapes that conform to common body parts and appendages that are common wart sites such as fingers, knuckles, crevices, toes and heels. Furthermore, and in conjunction with pre-cut shapes, the availability of occlusive adhesive articles with a corresponding release liner to ease preparation while limiting contact with skin oils and particulate debris would be very advantageous to the practical use of occlusive therapy.

Accordingly, there is not only a need for a less painful and less invasive therapy for wart removal, but also a more cosmetically acceptable, user friendly solution in a manner which is quick, easy, and effective. The development of the occlusive articles for wart treatment fulfills this need.

A search of the prior art did not disclose any patents that read directly on the claims of the instant invention; however, the following references were considered related.

The following patents disclose various methods, devices, and systems for wart treatment and related disorders:

U.S. Pat. No. 5,641,507, issued in the name of Devillez;

U.S. Pat. No. 6,593,371 B1, issued in the name of Staggs;

U.S. Pat. No. 5,702,694, issued in the name of Chamness;

U.S. Pat. No. 6,355,251 B1, issued in the name of Zamoyski;

U.S. Pat. No. 6,403,123 B1, issued in the name of Scott et al.;

U.S. Pat. No. 6,020,005, issued in the name of Weathers;

U.S. Pat. No. 6,384,295 B2, issued in the name of Wehde; and

U.S. Pat. No. 5,476,664, issued in the name of Robinson et al.

U.S. Pat. No. 5,310,402, issued in the name of Rollband discloses a tape for attaching bandages, with non-adhesive tabs at each end for easy manipulation and removal while wearing gloves.

U.S. Pat. No. 5,637,080, issued in the name of Geng discloses an external wound dressing having a reversibly movable cover.

U.S. Pat. No. 6,214,915 B1, issued in the name of Avakian et al. discloses stabilized thermoplastic compositions.

Web Site Publication, Duct Tape of Benefit in the Treatment of Verruca Vulgaris, University of Mich. Department of Pediatrics, December 2002.

Consequently, a need has been identified for a less painful and invasive therapy for wart removal that is convenient and specifically designed for the application of occlusive therapy.

### SUMMARY OF THE INVENTION

Therefore, it is an object of the present invention to provide an occlusive article for wart treatment in a pre-cut shape and size to assist application and conformation to anatomical body parts and appendages that commonly harbor warts with or without an adhesive coating to fix the occlusive article in position.

It is another object of the present invention to provide an occlusive article for wart therapy in cosmetic tints and shades that mimic skin tones and minimize the visual detection of therapy by casual observers.

It is another object of the present invention to provide a release liner for occlusive articles with adhesives to aid in the physical application and minimize contamination of the adhesive with inadvertent skin oil and debris contact.

It is another object of the present invention to provide a method for die cutting the occlusive articles and release liners together and packaging them as independent units.

It is another object of the present invention to provide an assortment of shapes and sizes to make selection of the appropriate shape and size optional to the end user.

It is another object of the present invention to provide occlusive articles for wart therapy in a continuous roll.

It is another object of the present invention to provide a dispenser for the occlusive articles in a continuous roll.

It is another object of the present invention to provide occlusive articles for wart therapy as a part of a kit with other synergistic therapies.

It is another object of the present invention to provide occlusive articles with a secondary adhesive backing to augment the occlusion and fixation of the occlusive article to the treatement area.

Briefly described according to one embodiment of the present invention, an occlusive article for wart treatment is disclosed, comprised of an occlusive component adapted to prevent moisture loss or limit moisture loss which provides an environment for the effective treatment of any wart where occlusive therapy may be practically administered. The occlusive article is formed of a flexible substrate material, such as occlusive tape with an adhesive for the purpose of attaching the article to the effected area.

A removable release liner covers the outer side of the adhesive portion of the occlusive article in order to afford protection thereto until desired use and to ease handling when administering.

The occlusive article is manufactured in various die-cut shapes, including but not limited to circular, square, rectangular, triangular, pentagonal, hexagonal, octagonal, trapezoidal, rhomboidal, hour glass, and elliptical in order to facilitate ease in consumer use and to accommodate consumer preferences.

The occlusive article is further provided in various flesh-colored tones via a color matching means in order to closely match and blend with a variety of skin pigments.

A kit is provided which contains an occluding or partially occluding means, such as a stack of individually-wrapped occlusive articles, synergistic therapies for wart resolution and instructions.

In an alternate embodiment briefly described according to the present invention, an occlusive article for wart treatment is disclosed, comprised of an occlusive component adapted to prevent moisture loss or limit moisture loss which provides an environment for the effective treatment of any wart where occlusive therapy may be practically administered. The occlusive article is formed of a flexible substrate material, such as occlusive tape with an adhesive for the purpose of attaching the article to the effected area.

The occlusive article is manufactured as a continuous roll in a continuous width including but not limited to 0.1 to 3.0 inches to facilitate ease in consumer use and to accommodate various wart sizes.

The occlusive article is further provided in various flesh-colored tones via a color matching means in order to closely match and blend with a variety of skin pigments.

A kit is provided which contains an occluding or partially occluding means, such as a roll of occlusive articles, synergistic therapies for wart resolution and instructions.

### BRIEF DESCRIPTION OF THE DRAWINGS

The advantages and features of the present invention will become better understood with reference to the following more detailed description and claims taken in conjunction with the accompanying drawings, in which like elements are identified with like symbols, and in which:

FIG. 1 is a perspective view of an occlusive article with an adhesive coated on one side for wart treatment according to the preferred embodiment of the present invention;

FIG. 2 is a perspective view of the back of a human hand shown inflicted with warts;

FIG. 3 is a perspective view of the flexible material forming the occlusive article according to the preferred embodiment of the present invention;

FIG. 4 is a perspective view of the occlusive article shown placed over a wart and further shows an occlusive article being applied to a wart to be treated;

FIG. 5 is a perspective view of the occlusive article shown fabricated in accordance with the present invention, in its assembled form prior to actual usage according to the preferred embodiment of the present invention;

FIG. 6 is a perspective view of a kit provided in accordance with the principles of the present invention for treating a wart; and

FIG. 7 is a front side elevation view of the occlusive article dispenser.

### DESCRIPTION OF THE PREFERRED EMBODIMENT

1. Detailed Description of the Figures

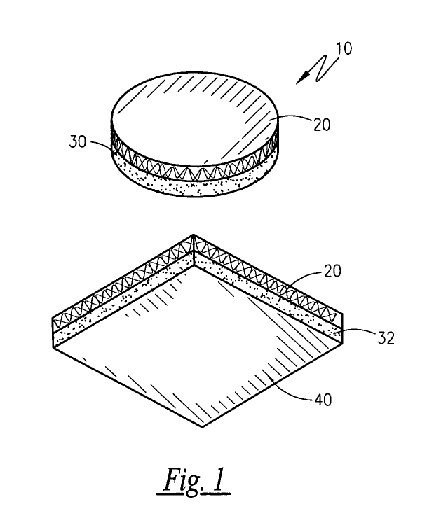

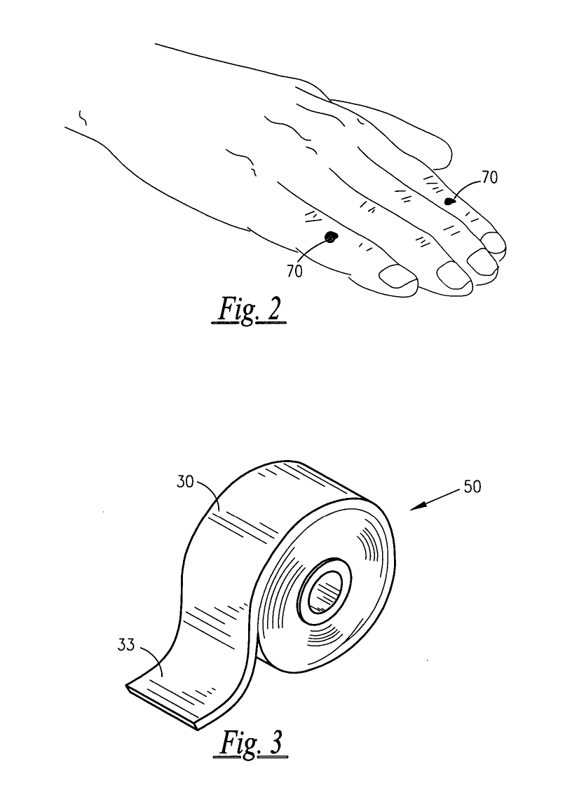

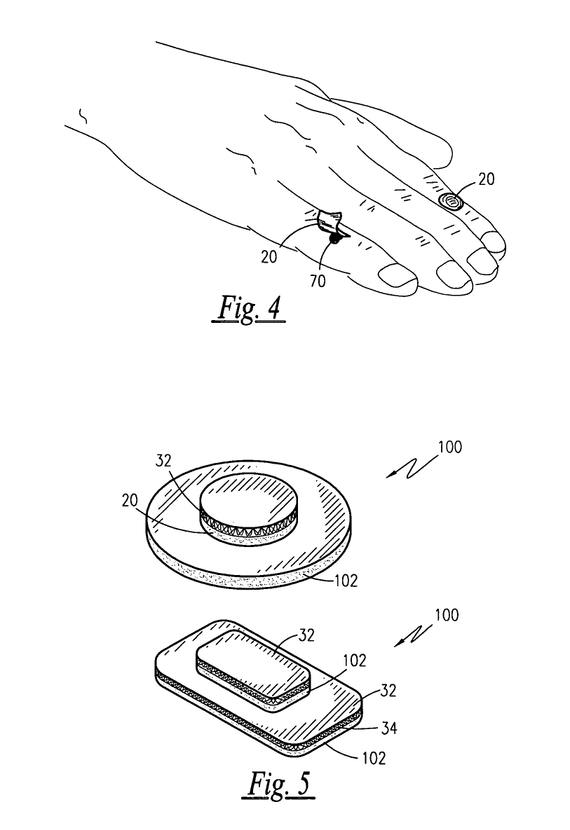

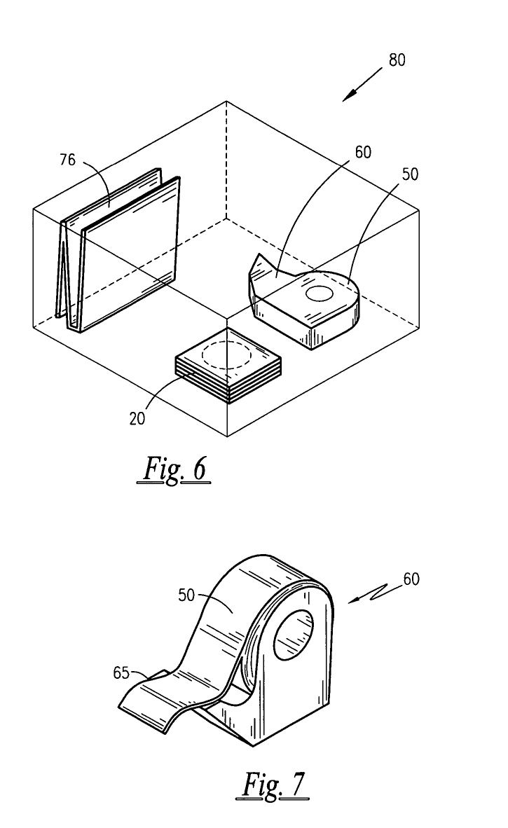

Referring now to FIGS. 1-5, occlusive article for wart treatment 10 is shown, according to the present invention, comprised of an occlusive article 20 adapted to provide an occlusive environment for the effective treatment of Verruca vulgaris (the common wart).

The occlusive article 20 is defined of a flexible material 30, for example occlusive or partially occlusive tape, film, sheet, or comparable backing. The occlusive article 20 is fabricated in a pre-cut shape and size to assist application and conformation to anatomical body parts and appendages that commonly harbor warts 70. Pre-cut shaped and sized occlusive articles 20 are provided on a readily peelable, single card or comparable backing as a plurality of occlusive article 20 units. As part of a final commercial product, the pre-cut shaped and sized occlusive articles 20 are packaged independently as a single card provided with a plurality of occlusive articles 20, or packaged with a plurality of cards, each card of the plurality of cards being provided with a plurality of occlusive articles 20. The pre-cut shaped and sized occlusive articles 20 are commercially available as an assortment of shapes and sizes in order to make selection of an appropriate shape and size optional to end user. Thus, the occlusive article 20 is manufactured in various die-cut shapes which include, but is not limited to circular, square, rectangular, triangular, pentagonal, hexagonal, octagonal, trapezoidal, rhomboidal, hour glass, and elliptical. The occlusive article 20 is manufactured utilizing suitable cutting die processing equipment (not shown) to facilitate ease in consumer use and to accommodate consumer preferences.

The occlusive article is further provided in various cosmetic tints and shades which mimic skin tones via a color matching means (not shown) in order to closely match and blend with a variety of skin pigments. A suitable color matching means known in the art is selected for coloring the occlusive articles 20 which minimizes visual detection of therapy by casual observers.

The occlusive article 20 further comprises an adhesive coating 32 disposed on an outer side of occlusive article 20 which functions as an occlusion or partial occlusion providing layer. The adhesive coating 32 contributes to occlusion through its inherent occlusive properties.

The occlusive article 20 is further defined of a single substrate 33 or multi-ply substrate 34 adapted to adhesively attach to an effected area. It is envisioned that the single substrate 33 or multi-ply substrate 34 is adapted with the adhesive coating 32 to facilitate adhesive attachment to an effected area.

A removable release liner 40 is included for embodiments incorporating an adhesive coating 32. The removable release liner 40 covers an outer side of the adhesive coating 32 in order to afford protection thereto until desired use. The removable release liner 40 is readily peelable from the adhesive coating 32. The removable release liner 40 aids in physical application of the occlusive article 20 and minimizes contamination of the adhesive coating 32 with inadvertent skin oil and debris contact. Occlusive article 20 embodiments incorporating the adhesive coating 32 and removable release liner 40 are fabricated in pre-cut shape and size and made available as individual units or in plurality using a die-cutting method. The individual units are packaged independently as a unit whether part of a final commercial package containing a plurality of units or sold individually.

Referring more specifically to FIG., the occlusive article 20 is shown provided as a continuous roll 50 of occlusive articles 20. The roll 50 includes a quantity of individual occlusive articles 20, each being easily severed from the roll 50. In order to facilitate effortless severance by each occlusive article 20 from the roll 50, a perforated severing line 52 is envisioned. The roll 50 of occlusive articles 20 defines a pre-cut width and a continuous length to promote easy dispensing of clinically relevant sized occlusive articles 20 for anatomical body parts and appendages that commonly harbor warts 70. The roll 50 of occlusive articles 20 has a slit width measuring approximately 0.10 inches to 3.0 inches.

The occlusive articles 20 of the roll 50 of occlusive articles 20 are each further defined as an occlusive or partially occlusive tape, film, sheet, or comparable backing. The occlusive articles 20 of the roll 50 of occlusive articles 20 are formed of a single substrate 33 or multi-ply substrate 34 adapted to adhesively attach to an effected area. It is envisioned that the single substrate 33 or multi-ply substrate 34 is adapted with an adhesive coating 32 to facilitate adhesive attachment to an effected area. The adhesive coating 32 contributes to occlusion through its inherent occlusive properties. A removable release liner 40 is also included for these embodiments incorporating the adhesive coating 32. The removable release liner 40 covers an outer side of the adhesive coating 32 and aids in the physical application of occlusive articles 20 and minimizes contamination of the adhesive coating 32 with inadvertent skin oil and debris contact.

The continuous roll 50 of occlusive articles 20 is further provided in various cosmetic tints and shades which mimic skin tones via a color matching means (not shown) in order to closely match and blend with a variety of skin pigments. A suitable color matching means known in the art is selected for coloring the roll 50 of occlusive articles 20 which minimizes visual detection of therapy by casual observers.

Referring now to FIG., a portable, lightweight dispenser 60 is provided for containing a continuous roll 50 of occlusive articles 20 in order to facilitate ease of retrieving or dispensing a single occlusive article 20 at a time. The dispenser 60 is adapted to house the continuous roll 50 of occlusive articles 20 in a suspended, rotational manner so as to allow individual occlusive articles 20 to be easily unrolled and dispensed therefrom. It is envisioned that the dispenser 60 is provided with a severing edge 65 to facilitate easy separation of a single occlusive article 20 from the roll 50 of occlusive articles 20 along the perforated severing line 52 thereof.

Referring now to FIGS., a multicomponent occlusive article 100 is provided. The multicomponent occlusive article 100 is comprised of an occlusive article 20 fabricated in a pre-cut shape and size affixed to a secondary adhesive tape 102 or comparable backing ultimately cut to a final size and shape. The multicomponent occlusive article 100 is adapted to augment occlusion and fixation of the occlusive article 20 thereof to anatomical body parts and appendages commonly harboring warts 70.

The occlusive article 20 of the multicomponent occlusive article 100 is defined of a flexible material, for example occlusive or partially occlusive tape, film, sheet, or comparable backing. The occlusive article 20 further comprises an adhesive coating 32 disposed on an outer side of occlusive article 20 to facilitate attachment of the occlusive article 20 to an effected area. The adhesive coating 32 contributes to occlusion through its inherent occlusive properties.

The occlusive article 20 of the muliticomponent occlusive article 100 is further defined of a single substrate 33 or a multi-ply substrate 34 adapted to adhesively attach to an effected area. It is envisioned that the single substrate 33 or multi-ply substrate 34 is adapted with the adhesive coating 32 to facilitate adhesive attachment to an effected area. The pre-cut shaped and sized occlusive articles 20 and secondary adhesive tape 102 or comparable backing include a removable release liner, wherein pre-cut shaped and sized occlusive articles 20 and secondary adhesive tape 102 incorporating the removable release liner 40 are made available as individual units using a die-cutting method or in plurality to aid in physical application of the multicomponent occlusive article 100 and to minimize contamination of adhesive coating 32 with inadvertent skin oil and debris contact. The individual units are packaged independently as a unit whether part of a final commercial package or sold individually.

The multicomponent occlusive article 100 is also provided on a readily peelable, single card or comparable backing as a plurality of occlusive article 20 units. As part of a final commercial product, the multicomponent occlusive article 100 is packaged independently as a single card provided with a plurality of occlusive articles 20, or packaged with a plurality of cards, each card of the plurality of cards being provided with a plurality of occlusive articles 20.

The pre-cut shaped and sized occlusive articles 20 of the multicomponent occlusive article 100 are commercially available as an assortment of shapes and sizes in order to make selection of an appropriate shape and size optional to end user.

The pre-cut shaped and sized occlusive articles and the secondary adhesive tape 102 or comparable backing of the multicomponent occlusive article 100 are available in various cosmetic tints and shades which mimic skin tones and minimize visual detection of therapy by casual observers.

Referring now to FIG., a kit 80 is provided for treating a wart 70 which comprises a package 82 for housing an occluding or partially occluding means, wherein occluding or partially occluding means is an occlusive article(s) 20. The kit 80 further comprises a continuous roll 50 of occlusive articles 20 and a multicomponent occlusive article 100. Synergistic therapies may also be provided as kit components which include but are not limited to anthralin, salicylic acid, lactic acid, glacial acetic acid, nitric acid, trichloracetic acid, and bichloroacetic acid. An instruction leaflet 76 is also provide in the kit 80.

Further in accordance with the present invention, a method is provided for treating one or more warts 70, wherein the method comprises the steps of removing the dispenser 60 from the package 82, severing at least one occlusive article 20 from the roll 50 of occlusive articles 20 using the severing edge 65 of dispenser 60, removing the release liner 40 from occlusive article 20, covering the wart 70 and skin area peripheral to the wart 70 with the outer side of occlusive article 20 lying in contact against the wart 70 so as to provide an occlusive or partially occlusive environment therefore maintaining the occlusive article 20 in position over wart 70 and skin area peripheral thereto for a predetermined period of time, removing the occlusive article 20 after the predetermined period of time, and repeating the aforementioned steps until a desired level of wart 70 removal has been acquired. The aforementioned method for treating one or more warts 70 may further include the step of debriding the wart 70 after removing the occlusive article 20 after the predetermined period of time, and then repeating the steps stated hereinabove.

2. Operation of the Preferred Embodiment

To use the present invention, user removes the occlusive article dispenser 60 from the package 82, severs at least one occlusive article 20 from the roll 50 of occlusive articles 20 using the severing edge 65 of dispenser 60, removes the release liner 40 from occlusive article 20, covers the wart 70 and skin area peripheral to the wart 70 with the outer side of occlusive article 20 lying in contact against the wart 70 so as to provide an occlusive or partially occlusive environment therefore. User then maintains the occlusive article 20 in position over wart 70 and skin area peripheral thereto for a predetermined period of time. User next removes the occlusive article 20 after the predetermined period of time, and repeats the aforementioned procedure until a desired level of wart 70 removal has been acquired.

The use of the present invention provides not only a less painful and invasive therapy for wart removal, but also a more cosmetically acceptable solution.

Therefore, the foregoing description is included to illustrate the operation of the preferred embodiment and is not meant to limit the scope of the invention. As one can envision, an individual skilled in the relevant art, in conjunction with the present teachings, would be capable of incorporating many minor modifications that are anticipated within this disclosure. The foregoing descriptions of specific embodiments of the present invention have been presented for purposes of illustration and description. They are not intended to be exhaustive or to limit the invention to the precise forms disclosed, and obviously many modifications and variations are possible in light of the above teaching. The embodiments were chosen and described in order to best explain the principles of the invention and its practical application, to thereby enable others skilled in the art to best utilize the invention and various embodiments with various modifications as are suited to the particular use contemplated. It is intended that the scope of the invention be defined by the Claims appended hereto and their equivalents. Therefore, the scope of the invention is to be broadly limited only by the following Claims.

What is claimed is:

1. An occlusive article for wart treatment comprising:
an occlusive article, said occlusive article is defined of a flexible material being fabricated in a pre-cut shape and size so as to form pre-cut shaped and sized occlusive articles particularly adapted to assist application and conformation to anatomical body parts and appendages that commonly harbor warts.

2. The occlusive article for wart treatment of claim 1, wherein said flexible material is formed of occlusive or partially occlusive tape, film, sheet, or comparable backing.

3. The occlusive article for wart treatment of claim 1, wherein said pre-cut shaped and sized occlusive articles are provided on a readily peelable, single card or comparable backing as a plurality of occlusive article units.

4. The occlusive article for wart treatment of claim 3,. wherein said pre-cut shaped and sized occlusive articles are packaged independently as a single card provided with a plurality of occlusive articles are packaged independently as a single card provided with a plurality of occlusive articles as part of a final commercial product, or packaged with a plurality of cards, wherein each card of said plurality of cards being provided with a plurality of occlusive articles as part of a final commercial product.

5. The occlusive article for wart treatment of claim 1, wherein said pre-cut shaped and sized occlusive articles are commercially available as an assortment of shapes and sizes in order to make selection of an appropriate shape and size optional to end user.

6. The occlusive article for wart treatment of claim 1, wherein said pre-cut shaped and sized occlusive articles are provided in various cosmetic tints and shades which mimic skin tones in order to closely match and blend with a variety of skin pigments, thereby minimizing visual detection of therapy by casual observers.

7. The occlusive article for wart treatment of claim 1, wherein said pre-cut shaped and sized occlusive articles further comprise an adhesive coating disposed on an outer side of each of said pre-cut shaped and sized occlusive articles as individual units or in a plurality of units, wherein said adhesive coating functions as an occlusion or partial occlusion providing layer, said adhesive coating contributes to occlusion through its inherent occlusive properties.

8. The occlusive article for wart treatment of claim 7, wherein said pre-cut shaped and sized occlusive articles further comprise a removable release liner, said removable release liner covers an outer side of said adhesive coating in order to aid in physical application of said occlusive article and minimize contamination of said adhesive coating with inadvertent skin oil and debris contact.

9. The occlusive article for wart treatment of claim 8, wherein said pre-cut shaped and sized occlusive articles are fabricated in pre-cut shape and size and made available as individual units using a die-cutting method.

10. The occlusive article for wart treatment of claim 9, wherein said individual units are packaged independently as a unit whether part of a final commercial package containing a plurality of units or sold individually.

11. The occlusive article for wart treatment of claim 1, wherein said occlusive article is defined of a single substrate or multi-ply substrate, wherein said single substrate or said multi-ply substrate is adapted to adhesively attach to an effected area.

12. The occlusive article for wart treatment of claim 11, wherein said single substrate or said multi-ply substrate is disposed with an adhesive coating to facilitate adhesive attachment to an effected area.

13. The occlusive article for wart treatment of claim 1, wherein said occlusive article is provided as a continuous roll of occlusive articles.

14. The occlusive article for wart treatment of claim 13, wherein said continuous roll of occlusive articles is contained within a portable, lightweight dispenser in order to facilitate ease of retrieving or dispensing a single occlusive article at a time.

15. An occlusive article for wart treatment comprising:
a continuous roll of occlusive articles, said continuous roll includes a quantity of individual occlusive articles, said continuous roll defines a pre-cut width and a continuous length to promote easy dispensing of clinically relevant sized occlusive articles for anatomical body parts and appendages that commonly harbor warts.

16. The occlusive article for wart treatment of claim 15, wherein said occlusive articles of said continuous roll are each defined as an occlusive or partially occlusive tape, film, sheet, or comparable backing.

17. The occlusive article for wart treatment of claim 15, wherein said occlusive articles of said continuous roll are formed of a single substrate or multi-ply substrate adapted to adhesively attach to an effected area.

18. The occlusive article for wart treatment of claim 15, wherein said single substrate or said multi-ply substrate is disposed with an adhesive coating to facilitate adhesive attachment to an effected area, said adhesive coating contributes to occlusion through its inherent occlusive properties.

19. The occlusive article for wart treatment of claim 18, wherein said single substrate or said multi-ply substrate further comprises a removable release liner, said removable release liner covers an outer side of said adhesive coating in order to aid in physical application of said occlusive articles and minimize contamination of said adhesive coating with inadvertent skin oil and debris contact.

20. The occlusive article for wart treatment of claim 15, wherein said continuous roll of occlusive articles is provided in various cosmetic tints and shades which mimic skin tones in order to closely match and blend with a variety of skin pigments, thereby minimizing visual detection of therapy by casual observers.

21. The occlusive article for wart treatment of claim 15, wherein said continuous roll of occlusive articles has a slit width measuring approximately 0.10 inches to 3.0 inches.

22. The occlusive article for wart treatment of claim 15, wherein said continuous roll of occlusive articles is contained within a portable, lightweight dispenser in order to facilitate ease of retrieving or dispensing a single occlusive article at a time.

23. An occlusive article for wart treatment comprising:
a multicomponent occlusive article, said multicomponent occlusive article is comprised of an occlusive article fabricated in a pre-cut shape and size affixed to a secondary adhesive tape or comparable backing ultimately cut to a final size and shape so as to form pre-cut shaped and sized occlusive articles, wherein said multicomponent occlusive article is adapted to augment occlusion and fixation of said occlusive article to anatomical body parts and appendages commonly harboring warts.

24. The occlusive article for wart treatment of claim 23, wherein said occlusive article of said multicomponent occlusive article is defined of a flexible material.

25. The occlusive article for wart treatment of claim 24, wherein said flexible material is formed of occlusive or partially occlusive tape, film, sheet, or comparable backing.

26. The occlusive article for wart treatment of claim 23, wherein said multicomponent occlusive article further comprises an adhesive coating, said adhesive coating is disposed on an outer side of said occlusive article of said multicomponent occlusive article in order to facilitate attachment of said occlusive article to an effected area, said adhesive coating contributes to occlusion through its inherent occlusive properties.

27. The occlusive article for wart treatment of claim 23, wherein said occlusive article of said multicomponent occlusive article is defined of a single substrate or a multi-ply substrate.

28. The occlusive article for wart treatment of claim 27, wherein said single substrate or said multi-ply substrate is disposed with an adhesive coating to facilitate adhesive attachment to an effected area.

29. The occlusive article for wart treatment of claim 23, wherein said pre-cut shaped and sized occlusive articles and said secondary adhesive tape or said comparable backing include a removable release liner.

30. The occlusive article for wart treatment of claim 29, wherein said pre-cut shaped and sized occlusive articles and said secondary adhesive tape or said comparable backing are made available as individual units using a die-cutting method or in plurality to aid in physical application of said multicomponent occlusive article and to minimize contamination of said adhesive coating with inadvertent skin oil and debris contact.

31. The occlusive article for wart treatment of claim 30, wherein said individual units are packaged independently as a unit whether part of a final commercial package or sold individually.

32. The occlusive article for wart treatment of claim 23, wherein said multicomponent occlusive article is provided on a readily peelable, single card or comparable backing as a plurality of occlusive article units.

33. The occlusive article for wart treatment of claim 23, wherein said multicomponent occlusive article is packaged independently as a single card provided with a plurality of occlusive articles, or packaged with a plurality of cards, wherein each card of said plurality of cards being provided with a plurality of occlusive articles as part of a final commercial product.

34. The occlusive article for wart treatment of claim 23, wherein said pre-cut shaped and sized occlusive articles are commercially available as an assortment of shapes and sizes in order to make selection of an appropriate shape and size optional to end user.

35. The occlusive article for wart treatment of claim 23, wherein said pre-cut shaped and sized occlusive articles are available in various cosmetic tints and shades which mimic skin tones and minimize visual detection of therapy by casual observers.

36. The occlusive article for wart treatment of claim 23, wherein said secondary adhesive tape or said comparable backing is available in various cosmetic tints and shades which mimic skin tones and minimize visual detection of therapy by casual observers.

37. A kit for treating a wart, comprising:
an occluding or partially occluding means;
a package, said package for housing said occluding or partially occluding means; and
an instruction leaflet.

38. The kit of claim 37, wherein said occluding or partially occluding means is an occlusive article(s).

39. The kit of claim 37, wherein said occluding or partially occluding means is a continuous roll of occlusive articles.

40. The kit of claim 37, wherein said occluding or partially occluding means is a multicomponent occlusive article(s).
